SEQUENCE LISTING (27 sequences)

SEQ ID NO: 1 - protein, 10 aa, Homo sapiens
PIQWNAPQPS

SEQ ID NO: 2 - protein, 11 aa, Homo sapiens
HPIQWNAPQPS

SEQ ID NO: 3 - protein, 9 aa, Homo sapiens
IQWNAPQPS

SEQ ID NO: 4 - protein, 10 aa, synthetic construct
  MOD_RES 10: note = Biotinylated via an optional linker
PIQWNAPQPS

SEQ ID NO: 5 - protein, 11 aa, synthetic construct
  MOD_RES 11: note = Biotinylated
PIQWNAPQPSK

SEQ ID NO: 6 - protein, 13 aa, synthetic construct
  MOD_RES 13: note = Bound to keyhole limpet hemocyanin
PIQWNAPQPSGGC

SEQ ID NO: 7 - protein, 10 aa, synthetic construct
LHDSNPYPRR

SEQ ID NO: 8 - protein, 11 aa, synthetic construct
  MOD_RES 11: note = Biotinylated
LHDSNPYPRRK

SEQ ID NO: 9 - protein, 6 aa, Homo sapiens
PIQWNA

SEQ ID NO: 10 - protein, 11 aa, synthetic construct
  MOD_RES 11: note = Biotinylated
TGLEPGIDYDK

SEQ ID NO: 11 - protein, 10 aa, Homo sapiens
TGLEPGIDYD

SEQ ID NO: 12 - protein, 31 aa, Homo sapiens
PSQPNSHPIQWNAPQPSHISKYILRWRPKNS

SEQ ID NO: 13 - protein, 31 aa, Mus musculus
PSQPNSHPIQWNAPEPSHITKYILRWRPKTS

SEQ ID NO: 14 - protein, 31 aa, Rattus sp.; identical to SEQ ID NO: 13

SEQ ID NO: 15 - protein, 31 aa, Bos taurus
PSQPNSHPIQWSAPESSHISKYILRWKPKNS

SEQ ID NO: 16 - protein, 16 aa, Mus musculus
RSSQSLVHSNGNTYLH

SEQ ID NO: 17 - protein, 7 aa, Mus musculus
KVSNRFS

SEQ ID NO: 18 - protein, 9 aa, Mus musculus
SQSTHVPYT

SEQ ID NO: 19 - protein, 5 aa, Mus musculus
DYEMH

SEQ ID NO: 20 - protein, 17 aa, Mus musculus
AIHPGRGAAAYNQKFKD

SEQ ID NO: 21 - protein, 13 aa, Mus musculus
SEEYGNYEDAMDY

SEQ ID NO: 22 - protein, 78 aa, Mus musculus
SSQSLVHSNGNTYLHWFLQKPGQSPKLLIYKVSNRFSGVPDRFSGSGSGTDFTLKISRVEAEDLGVYFCSQSTHVPYT

SEQ ID NO: 23 - protein, 81 aa, Mus musculus
DYEMHWVKQTPVHGLEWIGAIHPGRGAAAYNQKFKDKATLTADKSSSTAYMELSSLTSEDSAVYYCTRSEEYGNYEDAMDY

SEQ ID NO: 24 - protein, 112 aa, Mus musculus
DVVMTQTPLSLPVSLGDQASISCRSSQSLVHSNGNTYLHWFLQKPGQSPKLLIYKVSNRFSGVPDRFSGSGSGTDFTLKISRVEAEDLGVYFCSQSTHVPYTFGGGTKLEIK

SEQ ID NO: 25 - protein, 122 aa, Mus musculus
QVQLQQSGAELVRPGASVKLSCKALGYTLTDYEMHWVKQTPVHGLEWIGAIHPGRGAAAYNQKFKDKATLTADKSSSTAYMELSSLTSEDSAVYYCTRSEEYGNYEDAMDYWGQGTSVTVSS

SEQ ID NO: 26 - protein, 465 aa, Mus musculus
MEWSWVFLFLLSVTAGVQSQVQLQQSGAELVRPGASVKLSCKALGYTLTDYEMHWVKQTPVHGLEWIGAIHPGRGAAAYNQKFKDKATLTADKSSSTAYMELSSLTSEDSAVYYCTRSEEYGNYEDAMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 27 - protein, 238 aa, Mus musculus
MKLPVRLLVLMFWIPASSSDVVMTQTPLSLPVSLGDQASISCRSSQSLVHSNGNTYLHWFLQKPGQSPKLLIYKVSNRFSGVPDRFSGSGSGTDFTLKISRVEAEDLGVYFCSQSTHVPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

(19) United States
(12) Patent Application Publication
(10) Pub. No.: US 2025/0076314 A1
(43) Pub. Date: Mar. 6, 2025

(54) Anastellin Biomarker

(71) Applicant: Nordic Bioscience A/S, Herlev (DK)

(72) Inventors: Annika H. Hansen, København Ø (DK); Diana Julie Øersnes-Leeming, Klampenborg (DK); Morten Asser Karsdal, Kobenhavn Ø (DK); Jannie Marie Bülow Sand, Måløv (DK)

(73) Assignee: Nordic Bioscience A/S, Herlev (DK)

(21) Appl. No.: 18/753,275

(22) Filed: Jun. 25, 2024

Related U.S. Application Data
(63) Continuation-in-part of application No. 18/459,651, filed on Sep. 1, 2023.

(51) Int. Cl. G01N 33/68; C07K 16/18; G01N 33/577

(52) U.S. Cl. CPC G01N 33/6887; C07K 16/18; G01N 33/577; C07K 2317/34; G01N 2333/78; G01N 2470/10; G01N 2800/12

(57) ABSTRACT

Provided herein are monoclonal antibodies, assay kits and immunoassay methods for quantifying anastellin fragments with a N-terminus amino acid sequence PIQWNAPQPS (SEQ ID NO: 1) in a patient biofluid sample. Also provided are methods for detecting and/or for monitoring fibrotic diseases such as idiopathic pulmonary fibrosis (IPF) using the monoclonal antibodies.

### CROSS-REFERENCE TO RELATED APPLICATIONS

This application is a continuation-in-part of pending application U.S. Ser. No. 18/459,651, filed Sep. 1, 2023, the entirety of which is hereby incorporated by reference.

### SEQUENCE LISTING

A sequence listing is electronically submitted in XML format in compliance with 37 C.F.R. § 1.831(a) and is incorporated by reference herein. The XML file is named D7976CIPSEQ, was created on May 2, 2024 and is 27 KB in size.

### BACKGROUND OF THE INVENTION

### Field of the Invention

The present invention relates to a competitive immunoassay for detecting anastellin in a biological sample, and its use in identifying patients with conditions associated with idiopathic pulmonary fibrosis (IPF). The invention also relates to a kit for performing the competitive immunoassay.

### Description of the Related Art

IPF is a progressive interstitial lung disease with a significant impact on health-related quality of life and burden of disease in which life expectancy is 2-3 years from diagnosis if left untreated (1). Currently, only two antifibrotic treatment options are approved by the FDA, Nintedanib and Pirfenidone, where neither can halt the disease progression completely.

A hallmark of IPF is scarring of the lung interstitium by deposition and remodeling of the extracellular matrix (ECM) proteins (2). A ubiquitous and abundant component of the ECM is the high-molecular weight, dimeric ˜500-600 kDa glycoprotein fibronectin (FN) that consists of three domain units overall; 12 type I, 2 Type II, and 15-17 Type III domains (3). Each domain is a module of two anti-parallel β-sheets in which the Type III domain can extend and partially unfold when external force is applied from the surrounding ECM and cellular milieu (4). Upon mechanical stretch of FN, cryptic domains can be exposed and become available for protein interactions or cleavage. Anastellin is part of the cryptic domain in the first Type III domain and has been indicated as a site with bioactivity (5). FN stretch and resulting exposure of anastellin to allow interaction with other FN molecules has been shown to induce FN fibrillogenesis, generating the so-called superfibronectin that was found to have enhanced adhesivity (6). Furthermore, the anastellin peptide fragment has been shown to selectively inhibit lysophospholipid signaling by the Ras/ERK pathway (7). [0005] Accordingly, it has been shown that anastellin can inhibit tumor growth and metastasis in preclinical rodent models of human cancer and inhibit proliferation of endothelial cell microvessels in vitro (8-10). In spite of the effect anastellin has on FN fibrillogenesis, the role of anastellin in fibrosis is largely unknown and still needs to be investigated. In addition, associations of circulating anastellin with disease status or clinical parameters has not been investigated previously.

The applicant has designed a specific competitive immunoassay, FN-ANA, which utilizes the neoepitope of anastellin. The levels of the anastellin derived neoepitope can be used to evaluate patients with fibrosis, in particular IPF.

### SUMMARY OF THE INVENTION

The present invention is directed to a monoclonal antibody that specifically recognizes and binds to a peptide which is a neoepitope of the N-terminal of anastellin within a domain of the FN protein. Preferably, said neoepitope is comprised in amino acid sequence NH₂-PIQWNAPQPS (SEQ ID NO: 1). Thus, in a first aspect, the present invention provides a monoclonal antibody that specifically binds to the free N-terminus amino acid sequence PIQWNAPQPS (SEQ ID NO: 1) (i.e. the FN-ANA target sequence).

In a second aspect, the present invention also provides an immunoassay for detecting in a biological sample anastellin (FN-ANA). The method comprises contacting a patient sample with a monoclonal antibody that specifically binds to a neoepitope of anastellin and detecting and determining the amount of binding between the monoclonal antibody and peptides in the sample, wherein the neoepitope consists of an N-terminus sequence of anastellin.

In a preferred embodiment, the method of immunoassay comprises;
i) contacting a patient sample with a monoclonal antibody that specifically binds to the N terminus amino acid sequence PIQWNAPQPS (SEQ ID NO: 1) (also referred to herein as “FN-ANA”, or the “FN-ANA target sequence”); and
ii) detecting and determining the amount of binding between said monoclonal antibody and peptides in the sample.

In a preferred embodiment, the method of immunoassay is a method of immunoassay for detecting and/or monitoring IPF or a particular level of severity thereof in a patient, the method further comprising correlating said amount of binding with values associated with normal healthy subjects and/or values associated with known disease severity and/or values obtained from said patient at a previous time point and/or with a predetermined cut-off value.

In a preferred embodiment, the patient sample is a human biofluid sample. Preferably the sample is a blood-based sample, such as blood (whole blood), plasma or serum.

In a third aspect, the present invention provides a method of treating fibrosis, especially IPF in a patient in need thereof, the method comprising:
(a) carrying out a method of immunoassay for detecting fibrosis or a particular level of severity thereof in accordance with the first aspect of the present invention on a sample from a patient; and
(b) administering to the patient a therapy for the treatment of fibrosis if it is determined in step (a) that the patient has fibrosis or said particular level of severity thereof.

In a fourth aspect, the present invention is directed further to a kit for use in the immunoassay as described herein. The kit comprises a monoclonal antibody as described above.

### BRIEF DESCRIPTION OF THE DRAWINGS

FIG. 1 shows the sequence alignment analysis for FN-ANA between species. Arrows indicate cleavage site and start of the FN-ANA neoepitope for different species, letters in italics and underlined indicate the first 10 amino acids from the neoepitope start and bold letters highlight differences between human and other species investigated.

FIG. 2 shows FN-ANA neoepitope specificity. The mAb raised towards the FN-ANA neoepitope was specific towards the intended antigen (selection peptide with the neoepitope sequence) and the assay did not react with the truncated, elongated, nonsense coating, or nonsense selection peptides at different concentrations. The specificity of the immunoassay was investigated with colorimetric detection and the optical density (OD) was measured at wavelength 450 nm with 650 nm as background reference.

FIGS. 3A-3D characterize FN-ANA in idiopathic pulmonary fibrosis (IPF) subjects (FIGS. 3A-3B) and healthy control subjects (FIGS. 3C-3D). FIGS. 3A and 3C are scatterplots of age-related association of FN-ANA serum levels and FIGS. 3B and 3D are violin plots of comparison of FN-ANA serum levels in female and male sexes. Correlations were investigated by Spearman's rank correlation coefficient and comparison between groups were performed by Mann-Whitney U test. CI, confidence interval; ns, non-significant.

FIGS. 4A-4D show head-to-head comparison of FN-ANA versus FN-EDB in healthy controls and idiopathic pulmonary fibrosis (IPF) subjects. FIG. 4A is a violin plot of FN-ANA serum levels in healthy control and IPF subject groups, p<0.0001. FIG. 4B is ROC curve of FN-ANA in healthy control vs IPF subjects. FIG. 4C is a violin plot of FN-EDB serum levels in healthy control and IPF subject groups, p<0.05. FIG. 4D is a ROC curve of FN-EDB in healthy control vs IPF subjects. AUC, area under the curve; CI, confidence interval; ROC, receiver operating characteristics.

FIGS. 5A-5D characterise FN-ANA and FN-EDB in relation to IPF clinical parameters. FIG. 5A is a boxplot of FN-ANA serum levels in IPF subjects receiving or not receiving antifibrotic treatment, p<0.05. FIG. 5B is a scatterplot of FN-ANA serum levels and forced vital capacity (FVC; % predicted). FIG. 5C is a boxplot of FN-EDB serum levels in IPF subjects receiving or not receiving antifibrotic treatment. FIG. 5D is a scatterplot of FN-EDB serum levels and FVC (% predicted).

### DETAILED DESCRIPTION OF THE INVENTION

In this specification, unless expressly otherwise indicated, the word ‘or’ is used in the sense of an operator that returns a true value when either or both of the stated conditions is met, as opposed to the operator ‘exclusive or’ which requires that only one of the conditions is met. The word ‘comprising’ is used to mean ‘including or consisting of’.

A monoclonal antibody suitable for use in the method of the invention is disclosed herein and is specifically reactive with a neoepitope of anastellin, said neoepitope being comprised in the N-terminal amino acid sequence PIQWNAPQPS (SEQ ID NO:1). Where the monoclonal antibody is a monoclonal antibody that specifically binds to the N-terminus amino acid sequence PIQWNAPQPS (SEQ ID NO: 1), the monoclonal antibody preferably does not substantially recognize or specifically bind to a peptide having the N-terminus amino acid sequence HPIQWNAPQPS (SEQ ID NO: 2) (i.e. an elongated version of the FN-ANA target sequence extended at its N-terminus by the addition of a histidine residue) and/or does not substantially recognize or specifically bind to a peptide having the N-terminus amino acid sequence IQWNAPQPS (SEQ ID NO: 3) (i.e. a truncated version of the FN-ANA target sequence truncated by removal of the first proline residue). Preferably, the ratio of the affinity of said antibody for the FN-ANA target sequence to the affinity of said antibody for the elongated version and/or truncated version of the target sequence is at least at least 10 to 1, preferably at least 100 to 1, more preferably at least 1,000 to 1, more preferably at least 10,000 to 1, more preferably at least 100,000 to 1, and most preferably at least 1,000,000 to 1.

The term “specifically bind” as used herein means that the antibody binding is selective for the antigen (i.e., epitope) and that this binding can be distinguished from unwanted or non-specific interactions. The ability of a monoclonal antibody to bind to a specific epitope or peptide sequence can be measured either through an enzyme-linked immunosorbent assay (ELISA) as described herein or other techniques familiar to one of skill in the art, e.g. surface plasmon resonance (SPR) technique (analyzed e.g. on a BIAcore instrument) and traditional binding assays. The extent of binding of a monoclonal antibody to an unrelated protein is less than about 10% of the binding of the monoclonal antibody to the epitope or peptide as measured, e.g., by ELISA. “Affinity” refers to the strength of the sum total of non-covalent interactions between a single binding site of a molecule (e.g., an epitope binding region of an antibody) and its binding partner (e.g., an epitope or antigen). Unless indicated otherwise, as used herein, “binding affinity” refers to intrinsic binding affinity which reflects a 1:1 interaction between members of a binding pair (e.g., an antigen binding moiety and an antigen). The affinity of a molecule for its partner can generally be represented by the dissociation constant (Kd), which is the ratio of dissociation and association rate constants (k_{off} and kₒₙ, respectively). Thus, equivalent affinities may comprise different rate constants, as long as the ratio of the rate constants remains the same. The dissociation constant represents the concentration of the antigen at which half of the binding sites on the antibody are occupied. A lower Kd indicates a higher binding affinity between the antibody and antigen, while a higher Kd reflects weaker binding. Several methods are available to measure the Kd of an antibody, including surface plasmon resonance (SPR), isothermal titration calorimetry (ITC), and fluorescence-based assays. In certain aspects, a monoclonal antibody that binds to the epitope or peptide has a dissociation constant (Kd) of <1 pM, <100 nM, <10 nM, <1 nM, <0.1 nM, <0.01 nM, or <0.001 nM (e.g. 10⁸ M or less, e.g. from 10⁸ M to 10¹³ M, e.g., from 10⁹ M to 10¹³ M).

As used herein the term “monoclonal antibody” refers to both whole antibodies and to fragments thereof that retain the binding specificity of the whole antibody, such as for example a Fab fragment, F(ab′)2 fragment, single chain Fv fragment, or other such fragments known to those skilled in the art. As is well known, whole antibodies typically have a “Y-shaped” structure of two identical pairs of polypeptide chains, each pair made up of one “light” and one “heavy” chain. The N-terminal regions of each light chain and heavy chain contain the variable region, while the C-terminal portions of each of the heavy and light chains make up the constant region. The variable region comprises three complementarity determining regions (CDRs), which are primarily responsible for antigen recognition. The constant region allows the antibody to recruit cells and molecules of the immune system. Antibody fragments retaining binding specificity comprise at least the CDRs and sufficient parts of the rest of the variable region to retain said binding specificity. Antibodies which retain the same binding specificity may contain the same complementarity-determining regions (CDR). The CDR of an antibody can be determined using methods know in the art such as that described by Kabat et al.

Antibodies can be generated from B cell clones as described in the examples. The isotype of the antibody can be determined by ELISA specific for human IgM, IgG or IgA isotype, or human IgG1, IgG2, IgG3 or IgG4 subclasses. Other suitable methods can be used to identify the isotype.

The amino acid sequence of the antibodies generated can be determined using standard techniques. For example, RNA can be isolated from the cells, and used to generate cDNA by reverse transcription. The cDNA is then subjected to PCR using primers which amplify the heavy and light chains of the antibody. For example, primers specific for the leader sequence for all VH (variable heavy chain) sequences can be used together with primers that bind to a sequence located in the constant region of the isotype which has been previously determined. The light chain can be amplified using primers which bind to the 3′ end of the Kappa or Lamda chain together with primers which anneal to the V kappa or V lambda leader sequence. The full length heavy and light chains can be generated and sequenced.

Where the monoclonal antibody is a monoclonal antibody that specifically binds to the N-terminus amino acid sequence PIQWNAPQPS (SEQ ID NO: 1), the monoclonal antibody may for example be raised against a synthetic peptide having the N-terminus amino acid sequence PIQWNAPQPS (SEQ ID NO: 1). For example, the monoclonal antibodies may be raised by: (a) immunizing a rodent (or other suitable mammal) with a synthetic peptide comprising the N-terminus sequence PIQWNAPQPS (SEQ ID NO: 1), which peptide may optionally be linked at its C-terminus to an immunogenic carrier protein (such as keyhole limpet hemocyanin); (b) isolating and cloning a single antibody producing cell; and (c) assaying the resulting monoclonal antibodies to ensure that they have the desired specificity. An exemplary protocol of the development, production and characterization of suitable monoclonal antibodies is described in the Examples section, infra.

In certain exemplary embodiments, where the monoclonal antibody is a monoclonal antibody that specifically binds to the N terminus amino acid sequence PIQWNAPQPS (SEQ ID NO: 1), the monoclonal antibody may preferably comprise one or more complementarity-determining regions (CDRs) selected from:

| | CDR-L1: |
|---|---|
| (SEQ ID NO: 16) | |
| | RSSQSLVHSNGNTYLH, |
| | CDR-L2: |
| (SEQ ID NO: 17) | |
| | KVSNRFS, |
| | CDR-L3: |
| (SEQ ID NO: 18) | |
| | SQSTHVPYT, |
| | CDR-H1: |
| (SEQ ID NO: 19) | |
| | DYEMH, |
| | CDR-H2: |
| (SEQ ID NO: 20) | |
| | AIHPGRGAAAYNQKFKD, |
| | and |
| | CDR-H3: |
| (SEQ ID NO: 21) | |
| | SEEYGNYEDAMDY. |

Preferably the monoclonal antibody comprises at least 2, 3, 4, 5 or 6 of the above listed CDR sequences.

Preferably the monoclonal antibody has a light chain variable region comprising the CDR sequences:

| | CDR-L1: |
|---|---|
| (SEQ ID NO: 16) | |
| | RSSQSLVHSNGNTYLH |
| | CDR-L2: |
| (SEQ ID NO: 17) | |
| | KVSNRFS |
| | and |
| | CDR-L3: |
| (SEQ ID NO: 18) | |
| | SQSTHVPYT. |

Preferably the monoclonal antibody has a light chain that comprises framework sequences between the CDRs, wherein said framework sequences are substantially identical or substantially similar to the framework sequences between the CDRs in the light chain sequence below (in which the CDRs are shown in bold and underlined, and the framework sequences are shown in italics):

(SEQ ID NO: 22)
RSSQSLVHSNGNTYLHWFLQKPGQSPKLLIYKVSNRFSGVPDRFSGSGSG
TDFTLKISRVEAEDLGVYFCSQSTHVPYT

Preferably the monoclonal antibody has a heavy chain variable region comprising the CDR sequences:

| | CDR-H1: |
|---|---|
| (SEQ ID NO: 19) | |
| | DYEMH |
| | CDR-H2: |
| (SEQ ID NO: 20) | |
| | AIHPGRGAAAYNQKFKD |
| | and |
| | CDR-H3: |
| (SEQ ID NO: 21) | |
| | SEEYGNYEDAMDY. |

Preferably the monoclonal antibody has a heavy chain that comprises framework sequences between the CDRs, wherein said framework sequences are substantially identical or substantially similar to the framework sequences between the CDRs in the heavy chain sequence below (in which the CDRs are shown in bold and underlined, and the framework sequences are shown in italics)

(SEQ ID NO: 23)
DYEMHWVKQTPVHGLEWIGAIHPGRGAAAYNQKFKDKATLTADKSSSTAY
MELSSLTSEDSAVYYCTRSEEYGNYEDAMDY

Preferably, the monoclonal antibody comprises the light chain variable region sequence:

(CDRs bold and underlined; Framework
sequences in italics) and/or the heavy
chain variable region sequence:
(SEQ ID NO: 24)
DVVMTQTPLSLPVSLGDQASISCRSSQSLVHSNGNTYLHWFLQKPGQSPK
LLIYKVSNRFSGVPDRFSGSGSGTDFTLKISRVEAEDLGVYFCSQSTHVP
YTFGGGTKLEIK
(CDRs bold and underlined; Framework
sequences in italics).
(SEQ ID NO: 25)
QVQLQQSGAELVRPGASVKLSCKALGYTLTDYEMHWVKQTPVHGLEWIGA
IHPGRGAAAYNQKFKDKATLTADKSSSTAYMELSSLTSEDSAVYYCTRSE
EYGNYEDAMDYWGQGTSVTVSS

As used herein, the framework amino acid sequences between the CDRs of an antibody are “substantially identical” or “substantially similar” to the framework amino acid sequences between the CDRs of another antibody if they have at least 70%, 80%, 90% or at least 95% similarity or identity. The similar or identical amino acids may be contiguous or non-contiguous. The framework sequences may contain one or more amino acid substitutions, insertions and/or deletions. Amino acid substitutions may be conservative, by which it is meant the substituted amino acid has similar chemical properties to the original amino acid. A skilled person would understand which amino acids share similar chemical properties. For example, the following groups of amino acids share similar chemical properties such as size, charge and polarity: Group 1 Ala, Ser, Thr, Pro, Gly; Group 2 Asp, Asn, Glu, Gln; Group 3 His, Arg, Lys; Group 4 Met, Leu, Ile, Val, Cys; Group 5 Phe Thy Trp.

A program such as the CLUSTAL program to can be used to compare amino acid sequences. This program compares amino acid sequences and finds the optimal alignment by inserting spaces in either sequence as appropriate. It is possible to calculate amino acid identity or similarity (identity plus conservation of amino acid type) for an optimal alignment. A program like BLASTx will align the longest stretch of similar sequences and assign a value to the fit. It is thus possible to obtain a comparison where several regions of similarity are found, each having a different score. Both types of analysis are contemplated in the present invention. Identity or similarity is preferably calculated over the entire length of the framework sequences.

As used herein, the term “N-terminus” refers to an N-terminal peptide sequence at the extremity of a polypeptide, i.e. at the N-terminal end of the polypeptide, and is not to be construed as meaning in the general direction thereof. As used herein, the term “C-terminus” refers to a C-terminal peptide sequence at the extremity of a polypeptide, i.e. at the C-terminal end of the polypeptide, and is not to be construed as meaning in the general direction thereof.

As used herein, the terms “peptide” and “polypeptide” are used synonymously. The antibody according to the first aspect of the invention is, in particular, suitable for use in carrying out the methods of immunoassay according to the second and third aspect of the invention. Preferred embodiments and features of the antibody for use in the immunoassay methods according to the second and third aspect will therefore be apparent from the above discussion of the preferred embodiments of the monoclonal antibody according to the first aspect.

The immunoassay can detect and determine the amount of binding between said monoclonal antibody and peptides in the sample. The amount of binding can be correlated with values associated with normal healthy subjects and/or values associated with known disease severity and/or values obtained from said patient at a previous time point and/or with a predetermined cut-off value.

In preferred embodiments the immunoassay is a competition assay or a sandwich assay. The immunoassay may, for example, be a radio-immunoassay or an ELISA. Such assays are techniques known to the person skilled in the art. Most preferably the immunoassay is a competitive ELISA.

As used herein the term “ELISA” (enzyme-linked immunosorbent assay) refers to an immunoassay in which the target peptide present in a sample (if any) is detected using antibodies linked to an enzyme, such as horseradish peroxidase or alkaline phosphatase. The activity of the enzyme is then assessed by incubation with a substrate generating a measurable product. The presence and/or amount of target peptide in a sample can thereby be detected and/or quantified. ELISA is a technique known to those skilled in the art.

As used herein the term, the term “competitive ELISA” refers to a competitive enzyme-linked immunosorbent assay and is a technique known to the person skilled in the art.

As used herein the term “sandwich immunoassay” refers to the use of at least two antibodies for the detection of an antigen in a sample, and is a technique known to the person skilled in the art.

Preferably, the immunoassay is used to quantify the amount of anastellin in a biofluid, wherein said biofluid may be, but is not limited to, serum, plasma, bronchoalveolar lavage fluid, sputum, urine, amniotic fluid, tissue supernatant or cell/tissue supernatant.

The human biofluid sample may be a sample from a human patient having medical signs or symptoms indicative of a fibrotic disease, in particular IPF.

As used herein the term “amount of binding” refers to the quantification of binding between the antibody and peptides in the patient sample. Said quantification may for example be determined by comparing the measured values of binding in the patient sample against a calibration curve produced using measured values of binding in standard samples containing known concentrations of a peptide to which the antibody specifically binds, in order to determine the quantity of peptide to which the antibody specifically binds in the patient sample. Any suitable analytical method can be used for measuring the amount of binding. For example, an ELISA method can be used in which spectrophotometric analysis is used to measure the amount of binding both in the patient samples and when producing the calibration curve.

As used herein the term “predetermined cut-off value” means an amount of binding that is determined statistically to be indicative of a high likelihood of a disease (e.g. IPF) or a particular severity thereof in a patient, in that a measured value of the target peptide in a patient sample that is at or above the statistical cut-off value corresponds to at least a 70% probability, preferably at least an 75% probability, more preferably at least an 80% probability, more preferably at least an 85% probability, more preferably at least a 90% probability, and most preferably at least a 95% probability of the presence of said disease or particular severity thereof.

As used herein, the term “values associated with normal healthy subjects” means standardised quantities of binding determined by the method described supra for samples from subjects considered to be healthy, i.e. without disease (i.e. without IPF); and the term “values associated with known disease severity” means standardised quantities of binding determined by the method described supra for samples from patients known to have disease (i.e. IPF) of a known severity.

In the specific assay disclosed herein which measures in biofluids target peptides having the N-terminus amino acid sequence PIQWNAPQPS (SEQ. ID No. 1), the calibration curve is produced using standard samples of known concentration of a calibration peptide having the N-terminus amino acid sequence PIQWNAPQPS (SEQ. ID No. 1), and which may in particular consist of the amino acid sequence PIQWNAPQPS (SEQ. ID No. 1). The values measured in the biofluid samples are compared to the calibration curve to determine the actual quantity of target peptide in the sample.

In a preferred embodiment, the monoclonal antibody may be labeled in order to determine the amount of binding of said monoclonal antibody.

Preferably, the monoclonal antibody may be an enzyme-linked antibody. The enzyme may be, but is not limited to, horseradish peroxidase (HRP).

Preferably, the monoclonal antibody may be radiolabeled or linked to a fluorophore.

Although these are preferred labels to be used with the invention, it is envisaged that any suitable labeling system may be employed, such as, but not limited to, DNA reporters or electrochemiluminescent tags.

Alternatively, a second labeled antibody which recognizes the first monoclonal antibody may be used to determine the amount of binding of said first monoclonal antibody. The further labeled antibody may be labeled using a label as described above.

In a preferred embodiment of the invention, the competitive immunoassay may further comprise correlating the quantity of anastellin determined by said method with standard fibrotic disease samples of known disease severity to evaluate the severity of a fibrotic disease. Such a fibrotic disease may be, but is not limited to, IPF.

The method may be an immunoassay method for diagnosing and/or monitoring and/or assessing the likelihood of an fibrotic disease in a patient, the method comprising carrying out the method of immunoassay of the second aspect of the invention on a biofluid sample obtained from said patient with the monoclonal antibody, detecting and determining the amount of binding between the monoclonal antibodies and peptides in the sample, and correlating said amount of binding with values associated with normal healthy subjects and/or values associated with known disease severity and/or values obtained from said patient at a previous time point. Preferably the fibrotic disease is IPF.

The method can be used to monitor the response of the patient to treatment. A sample can be taken once treatment has started, and analysed using the immunoassay of the second aspect. The amount of binding between the monoclonal antibodies and peptides in the sample can be correlated with values obtained from said patient at a previous time point, for example prior to treatment.

In a third aspect, the present invention provides a method of treating fibrosis, especially IPF in a patient in need thereof, the method comprising:
(a) carrying out a method of immunoassay for detecting fibrosis or a particular level of severity thereof in accordance with the second aspect of the present invention on a sample from a patient; and
(b) administering to the patient a therapy for the treatment of fibrosis if it is determined in step (a) that the patient has fibrosis or said particular level of severity thereof.

Preferred embodiments of the method in accordance with the third aspect will be apparent from the foregoing discussion of preferred embodiments of the methods according to the second aspect.

The therapy may be any therapy suitable for treating the fibrotic disease in question, such as IPF. The therapy may for example comprise or consist of one or more medicaments, one or more lifestyle changes, one or more surgeries or combinations thereof. Medicaments may be formulated for topical, inhaled, or systemic administration. Topical medicaments may for example be formulated as creams, foams, gels, lotions, or ointments for administration. Systemic medicaments may for example be formulated for enteral or parenteral administration. Surgeries may be curative surgeries, preventative surgeries, palliative surgeries and/or restorative surgeries. Inhaled medications include aerolised liquids or fine powders that can be administered using an metered dose device such as an inhaler, or a nebuliser.

Suitable treatments for IPF include Nintedanib and Pirfenidone.

In a fourth aspect, the present invention provides an immunoassay kit comprising a monoclonal antibody in accordance with the first aspect of the present invention, and at least one of:
a streptavidin coated well plate;
a biotinylated peptide PIQWNAPQPS-Li-Biotin (SEQ ID NO: 4) wherein Li is an optional linker;
a calibrator protein comprising the N-terminus amino acid sequence PIQWNAPQPS (SEQ ID NO: 1);
an antibody biotinylation kit;
an antibody HRP labelling kit; or
an antibody radiolabelling kit.

The immunoassay kit according to the fourth aspect of the invention is, in particular, suitable for use in carrying out the method of immunoassay according to the second and third aspects of the invention. Further preferred embodiments and features of the immunoassay kit according to the fourth aspect will therefore be apparent from the above discussion of the preferred embodiments of the methods according to the second and third aspect.

The kit may be for use in diagnosing or predicting the risk of a fibrotic disease, preferably in conjunction with the methods according to the second aspect of the invention. Preferably the fibrotic disease is IPF.

The invention is exemplified in the following examples which refer to the figures.

### Examples

### Materials and Methods

### Reagents and Peptides:

All reagents used were high quality chemicals from Merck (Whitehouse Station, NJ, USA) and Sigma (St. Louis MO, USA) unless otherwise specified. Synthetic peptides (Table 1) were obtained from Genscript (Piscataway, NJ, US), and were high-grade purity.

TABLE 1
Overview of synthetic peptides used for
assay development

| | |
|---|---|
| Immunogenic peptide | PIQWNAPQPS-GGC-KLH |
| | (SEQ ID NO: 6) |
| Selection peptide | PIQWNAPQPS (SEQ ID NO: 1) |
| Coating peptide | PIQWNAPQPS-K-Biotin |
| | (SEQ ID NO: 5) |
| Truncated peptide | IQWNAPQPS (SEQ ID NO: 3) |
| Elongated peptide | HPIQWNAPQPS (SEQ ID NO: |
| | 2) |
| Nonsense peptide | LHDSNPYPRR (SEQ ID NO: 7) |
| Nonsense coating | LHDSNPYPRR-K-Biotin |
| peptide | (SEQ ID NO: 8) |

### Identification of Neoepitope Target:

The first ten amino acids of the anastellin N-terminal within the FN protein, as stated at Uniprot.com (Identifier: PRO_0000390479), were considered a potential neoepitope in which the six first amino acids were expected to be important for antibody-antigen recognition. Thus, the initial six amino acids (PIQWNA) (SEQ ID NO: 9) were analysed for uniqueness by protein blasting using the NPS@: Network Protein Sequence Analysis tool (11), to ensure no cross-reaction with other proteins, followed by analysis for species homology using the UniProt sequence alignment tool (12). The potential neoepitope generated at the anastellin N-terminal was named FN-ANA and antibody production and subsequent assay development was initiated.

### Monoclonal Antibody Production and Selection:

Monoclonal antibody (mAb) was raised against the FN-ANA neoepitope sequence 6261627PIQWNAPQPS (SEQ ID NO: 1) using the following method. Five female Balb/C mice of 6-7 weeks of age were immunized subcutaneously with 200 μL emulsified antigen and 100 μg immunogenic peptide (PIQWNAPQPS-GGC-KLH (SEQ ID NO: 6)) using Stimmune (Thermo Fisher). The immunizations were repeated every second week until stable serum antibody titer levels were reached. The mice with the highest serum titer and showing best selectivity towards the selection peptide (PIQWNAPQPS (SEQ ID NO: 1)), and not the elongated peptide (HPIQWNAPQPS (SEQ ID NO: 2)), was chosen for fusion and rested for a month. Then, the selected mice were boosted intravenously with 50 μg immunogenic peptide in 100 μL 0.9% NaCl solution 3 days before isolation of the spleen for cell fusion. The mouse spleen cells were fused with SP2/0 myeloma cells to produce hybridoma cells as described by Gefter et al. (13). Hybridoma cells were plated in individual wells into 96-well microtiter plates for further growth using the limiting dilution method to promote monoclonal growth. An indirect ELISA performed on streptavidin-coated 96-well microtiter plates with a biotin-labelled peptide (PIQWNAPQPS-K-biotin (SEQ ID NO: 5)) as coating peptide was used to screen supernatants from hybridoma clones for reactivity to identify the best mAb-producing clones. The best clones were selected based on specificity to the selection peptide and no reaction to the deselecting peptides (elongated, truncated, and nonsense peptides; see Table 1). Supernatant was harvested and mAb was purified using HiTrap affinity columns (GE Healthcare Life Science, Little Chalfront, Buckinghamshire, UK) whereafter the mAb was labeled with HRP using the using the Roche Peroxidase Labeling Kit (cat. no. 11829696001, Merck). Finally, antibody isotype was determined using isotype-specific anti-sense primers or universal primers following the technical manual of SMARTScribe™ Reverse Transcriptase Kit. (Takara, Cat. No.: 639537). All procedures were performed according to the manufacturers' instructions.

The isotype, sequence and CDRs of this monoclonal antibody were determined. The sequence of the chains are as follows (CDRs underlined and in bold; N-terminus signal peptide and C-terminus Constant region in italics).

Heavy Chain Sequence (Mouse IgG1 isotype):
(SEQ ID NO: 26)
MEWSWVFLFLLSVTAGVQSQVQLQQSGAELVRPGASVKLSCKALGYTLTD
YEMHWVKQTPVHGLEWIGAIHPGRGAAAYNQKFKDKATLTADKSSSTAYM
ELSSLTSEDSAVYYCTRSEEYGNYEDAMDYWGQGTSVTVSSAKTTPPSVY
PLAPGSAAQTNSMVTLGCLVKGYFPEPVTVTWNSGSLSSGVHTFPAVLQS
DLYTLSSSVTVPSSTWPSETVTCNVAHPASSTKVDKKIVPRDCGCKPCIC
TVPEVSSVFIFPPKPKDVLTITLTPKVTCVVVDISKDDPEVQFSWFVDDV
EVHTAQTQPREEQFNSTFRSVSELPIMHQDWLNGKEFKCRVNSAAFPAPI
EKTISKTKGRPKAPQVYTIPPPKEQMAKDKVSLTCMITDFFPEDITVEWQ
WNGQPAENYKNTQPIMDTDGSYFVYSKLNVQKSNWEAGNTFTCSVLHEGL
HNHHTEKSLSHSPGK
Light Chain Sequence (Mouse Kappa Isotype):
(SEQ ID NO: 27)
MKLPVRLLVLMFWIPASSSDVVMTQTPLSLPVSLGDQASISCRSSQSLVH
SNGNTYLHWFLQKPGQSPKLLIYKVSNRFSGVPDRFSGSGSGTDFTLKIS
RVEAEDLGVYFCSQSTHVPYTFGGGTKLEIKRADAAPTVSIFPPSSEQLT
SGGASVVCFLNNFYPKDINVKWKIDGSERQNGVLNSWTDQDSKDSTYSMS
STLTLTKDEYERHNSYTCEATHKTSTSPIVKSFNRNEC.

### FN-ANA Assay Development:

The final FN-ANA assay parameters were determined empirically by several optimizing experiments where various reagents, concentrations, incubation time and temperature were investigated in addition to testing reactivity to selection and deselection peptides (Table 1) and human sera. The final FN-ANA competitive ELISA procedure was conducted with chemiluminescence detection and determined as follows: 100 μL of 3 ng/ml coating peptide (PIQWNAPQPS-K-biotin (SEQ ID NO: 5)) in coating buffer (50 mM tris-buffered saline (TBS) with 1% (w/v) bovine serum albumin, 0.1% (w/v) Tween-20, and 0.36% (v/v) Bronidox (BTB), 4 g/L NaCl, 0.20 g/L KCl, pH 8.0) was added to each well in a 96-well streptavidin-coated microtiter white plate (cat. No. 655995, Greiner Bio-one, Taufkirchen, Germany), whereafter it was incubated for 30 minutes at 20° C. with 300 RPM orbital shaking. After incubation, the plate was emptied and washed 5 times in washing buffer (consisting of 25 mM Tris, 3.0 g/L NaCl, 0.1% (w/v) Tween-20, pH 7.2). A 10-point calibration curve with 2-fold dilution was prepared from a start concentration of 200 ng/ml selection peptide (PIQWNAPQPS (SEQ ID NO: 1)) in incubation buffer (50 mM TBS-BTB, 4.0 g/L NaCl, 0.20 g/L KCl, 5% Liquid II (cat. No. 11941836001, Roche), pH 8.0). 20 μL/well of standards, assay controls, and serum samples were added to appropriate wells in double determinations followed by the addition of 100 μL/well of 6 ng/ml of HRP-labelled mAb in incubation buffer, whereafter plates were incubated at 4° C. for 20 hours with 300 RPM orbital shaking. The following day, chemiluminescence substrate (cat. No. 11582950001, Roche Diagnostics) was prepared according to manufacturer's instructions and mixed by rotation in the dark for 20 minutes at room temperature prior to plate analysis. Following a washing step, 100 L/well of chemiluminescence substrate was added, and the plate was incubated for a total of 3 minutes in darkness with 1 minute of 300 RPM orbital shaking before detection with luminescence on a SpectraMax i3x microplate reader (Molecular Devices, Sunnyvale CA, USA) at 1 second integration time at all wavelengths as microplate reader settings. Data analysis was conducted in SoftMax Pro version 7.1.0 software (Molecular Devices) with the 10-point calibration curve plotted using a 4-parameter logistic curve fit of the selection peptide standards.

### Technical Evaluation

Antibody specificity was evaluated using the synthetic peptides and determined as a percentage of signal inhibition of two-fold diluted selection and deselection peptides (elongated, truncated, and nonsense) in addition to a non-sense coating (peptide details are listed in Table 1) using a colorimetric detection method with 96-well streptavidin-coated microtiter plate (cat. No. 11940279, Roche Diagnostics, Hvidovre, Denmark) and using the above procedure with the following exceptions: Instead of adding chemiluminescent substrate, the colorimetric substrate 3,3′,5,5′-tetramethylbenzidine One (cat. No. 4380H, Kem-En-Tec, Taastrup, Denmark) was added to each well after the incubation step with the HRP-labeled FN-ANA antibody. The plate was incubated for 15 minutes with 300 RPM orbital shaking in darkness whereafter the incubation was stopped by adding 100 μL/well of 0.1% (v/v) sulphuric acid. The absorbance was measured at wavelength 450 nm with 650 as reference on a microplate reader (VersaMax, Molecular Devices, Sunnyvale, CA, USA).

Ten independent assay runs were used to establish the IC50 (half-maximal inhibition concentration) and the upper limit of quantification (ULOQ), determined as the highest concentration of selection peptide that attained a recovery percentage within 100±20% of the nominal concentration. The lower limit of quantification (LLOQ) of the analyte was determined for serum samples by triplicate assessment of five independent runs using four human serum samples that covered the lower range of the calibration curve. The samples' CV % with 95% confidence interval were plotted against the measured sample concentrations and a power regression model was used to estimate the LLOQ as the lowest analyte concentration with a CV % equal to 20%. The limit of blank (LOB) for the assay was established as the mean signal of 60 determinations of blank samples (i.e., incubation buffer) plus 3× standard deviation (SD). The intra- and inter-assay variations were determined from 10 independent runs of eight human serum samples and two assay control samples (i.e. selection peptide at a low and high concentration) with CV %≤15% as acceptance criteria. The minimum required dilution (MRD) and assay linearity of diluted samples was determined by two-fold dilutions of four human serum samples quantified in three independent runs, where recovery of diluted samples was calculated as percentage of recovery of the undiluted sample with recovery percentage within 100±20% as acceptance threshold for the dilution.

The stability of the analyte was determined in human serum samples stressed by up to five freeze/thaw cycles or by storage at 4° C. or 20° C. for 2, 4, 24, or 48 hours. Recovery was calculated with unstressed samples as reference. Assay accuracy of the analyte was determined in three human serum samples spiked with two-fold dilutions of the selection peptide and three human serum samples spiked with two-fold dilutions of another human serum sample with high known concentration of the analyte. Percentage recovery was calculated for the spiked samples using the theoretical total amount of analyte in the sample as reference. Analytical interference was evaluated by spiking a low/high concentration of lipids (1.5/5.0 mg/mL), haemoglobin (2.5/5.0 mg/mL), or biotin (5.0-100 ng/ml) into three human serum samples with known concentrations and calculating the recovery percentage in the spiked sample with the non-spiked sample as reference. All tests used double determinations of samples unless otherwise stated.

### Clinical Evaluation

The clinical relevance of the FN-ANA assay was evaluated in serum samples from 98 IPF and 133 healthy control subjects. Measurements of serum samples were performed in double determinations and values outside the measurements range were assigned the value of LLOQ (lower limit of quantification) or ULOQ (upper limit of quantification) as appropriate. Healthy controls were obtained from a commercial vendor (Discovery Life Science, Los Osos, CA). IPF patients had prevalent disease at the time of blood sample collection and were part of a larger study described previously (14). Inclusion criteria for IPF subjects were >18 years of age and an IPF diagnosis, and exclusion criteria were if linguistic or intellectual barriers prevented completion of questionnaires. Clinical parameters were collected at the same time of blood collection.

### Comparison of FN-ANA to a Different Assay Targeting the Type III Domain of FN:

The utility of FN-ANA was compared to a different immunoassay specific for another type III domain of FN, the EDB domain (FN-EDB). The FN-EDB immunoassay was compared head-to-head with FN-ANA, and the diagnostic abilities were evaluated in the IPF patient cohort (n=98) and healthy controls (n=133). Additionally, associations with clinical parameters were investigated and compared for the two assays. The methodical procedure of FN-EDB was similar to that of FN-ANA apart from the amino acid sequence of the synthetic peptides used for coating the plate (TGLEPGIDYD-K-Biotin (SEQ ID NO: 10)), assay controls (TGLEPGIDYD (SEQ ID NO: 11)), and standard peptide (TGLEPGIDYD (SEQ ID NO: 11)) to produce the calibration curve. In addition, the coating buffer (25 mM PBS-BTB, 8 g/L NaCl, pH 7.4) and incubation buffer (25 mM PBS-BTB, 8 g/L NaCl, 5% Liquid II (cat. No. 11941836001, Roche), pH 7.4) differed. The neoepitope-specificity of the mAb used in FN-EDB immunoassay was determined as 1324↓1325 TGLEPGIDYD (SEQ ID NO: 11) within the FN sequence.

### Statistical Analysis

Biomarker serum levels in IPF and healthy control subjects were analysed by Mann-Whitney U test. Separation of groups was assessed by the area under the curve (AUC) by receiver operations characteristics (ROC). Correlations of biomarker serum levels to clinical parameters were assessed by Spearman's rank correlation coefficient and statistical significance between sexes were assessed by Mann-Whitney U test. Recovery percentage (REC %), interquartile range (IQR) of biomarker measurements and 95% confidence interval (95% CI) of estimations are presented as appropriate. P-values below 0.05 were considered statistically significant. Statistical analysis and graphs were performed using GraphPad Prism version 9.5.0 (GraphPad Software, Inc., La Jolla, CA).

### Ethical Statement

All animals were treated in accordance with the guidelines for animal welfare. Antibody production in mice was approved by the Danish National Authority (The Animal Experiments Inspectorate) under approval number 2013-15-2934-00956. The collection and retrieval of the human serum complied with international ethical guidelines for handling human samples and patient information. All participants signed an informed consent, and the studies were approved by the local ethical committee and in compliance with the Helsinki Declaration of 1975.

### Results

### FN-ANA Technical Assessment and Characterization:

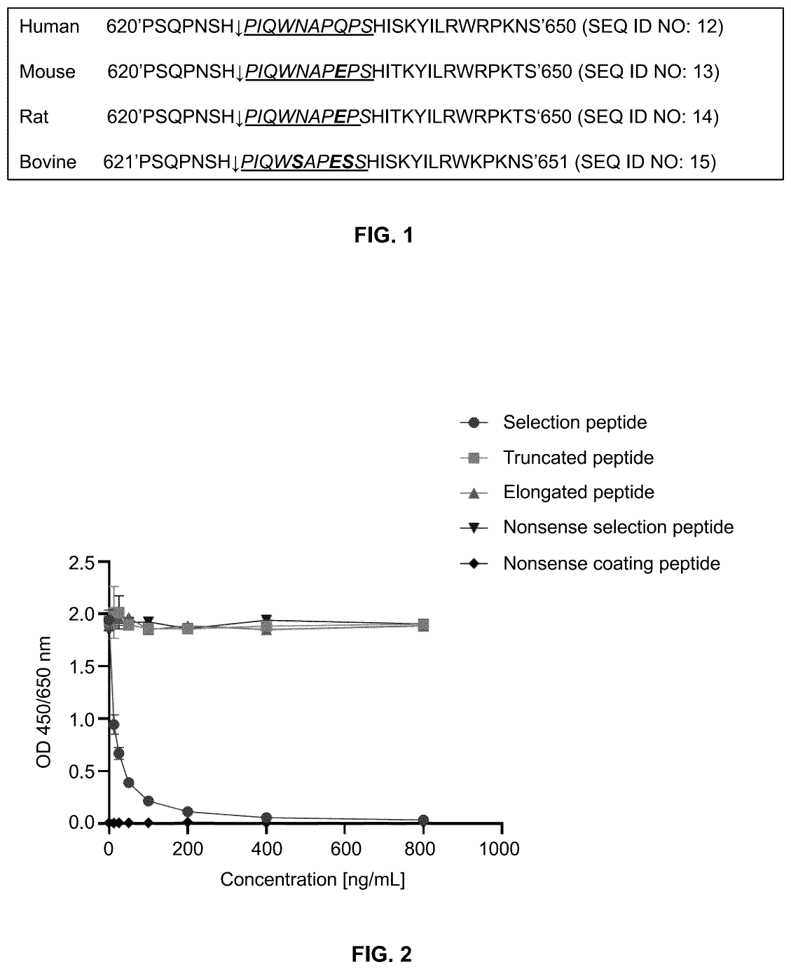

The initial six amino acids of the N-terminal of anastellin were investigated for uniqueness from other proteins and, based on the uniqueness, chosen as a neoepitope representing FN remodelling and anastellin release. In addition, sequence preservation of the anastellin N-terminal was investigated by comparison of the first 10 amino acids between human (SEQ ID NO: 12), mouse (SEQ ID NO: 13), rat (SEQ ID NO: 14), and bovine (SEQ ID NO: 15) (FIG. 1). The sequence had 100% preservation between all compared species for the first four amino acids with the first divergence at the fifth amino acid for the bovine sequence. The mouse and rat sequences had 100% preserved alignment from neoepitope start up until 7^{th} amino acid.

For FN-ANA mAb generation, mice were immunized with an immunogenic synthetic peptide with the first 10 amino acids from the neoepitope starting point of the human sequence. The resulting mAb with the highest selectivity for the selection peptide and the best native reactivity and stability was chosen for assay development. The FN-ANA assay was found to only react with the intended peptides (selection and coating peptide) and none of the deselection peptides (truncated, elongated, nonsense coating and nonsense peptides), indicating the mAb-antigen recognition of the intended neoepitope was highly specific (FIG. 2).

A summary of the technical evaluation of the final the FN-ANA assay is listed in Table 2. In brief, the LOB was determined to 1.47 ng/mL while the measurement range for analyte quantification in human serum was determined to be 1.6-100 ng/ml while achieving low intra-(4.1%) and inter-(9.3%) assay variation of analytical measurements. Linearity of analyte in serum had a mean recovery percentage within range of acceptance for 1:2 (102.5%) and 1:4 dilutions (110.0%), and good accuracy for analyte recovery when investigated by synthetic peptide recovery in serum (114.9%) and serum recovery in serum (105.4%). Analyte stability was accepted for at least five freeze-thaw cycles of serum samples (97.9%) and for prolonged storage of samples at different temperatures and times for up to 4 h at 20° C. (93.5%) and 48 h at 4° C. (90.6%), indicating preservation of neoepitope integrity. Interference was not observed for serum samples spiked with hemoglobin or lipids, or for up to 80 ng/ml of biotin.

TABLE 2
Technical summary of FN-ANA assay parameters

| Assay format | Competitive chemiluminescent | |
|---|---|---|
| | immunoassay; incubation 20 h | |
| | at 4° C. | |
| Measurement range: LLOQ-ULOQ | 1.6-100 | ng/mL |
| Evaluated matrix (MRD) | Human serum (undiluted) | |
| Intra-assay CV %, mean (min-max) | 4.1%, | (1.3-9.7%) |
| Inter-assay CV %, mean (min-max) | 9.3% | (3.3-19.5%) |
| LOB | 1.47 | ng/mL |
| Mean Slope of calibration curve | 1.07 | |
| IC50 | 8.86 | ng/mL |
| Curve fit model | 4-point logistic curve fit | |
| Linearity (mean REC % at 1:2/1:4 from | 102.5%/110.0% | |
| MRD) | | |
| Accuracy, mean REC % (IQR) | | |
| Peptide in serum | 114.9% | (108.6-125.5%) |
| Serum in serum | 105.4% | (97.2-114.6%) |
| Freeze-thaw cycles of serum samples, | 5 cycles, 97.9% (90.2-109.5%) | |
| mean REC % (IQR) | | |
| Accepted stress of serum samples, mean | | |
| REC % (IQR) | | |
| 48 h at 4° C. | 90.6% | (84.4-95.7%) |
| 2 h at 20° C. | 98.2% | (85.5-104.1%) |
| 4 h at 20° C. | 93.5% | (78.6-106.2%) |
| Interference | | |

| | |
|---|---|
| Hemoglobin (REC %, low/high) | 109.2%/110.4% |
| Lipids (REC %, low/high) | 92.2%/89.2% |
| Biotin (REC %, low/mid/high) | 103.1%/91.2%/83.2% |

REC % within 100 ± 20% was regarded as acceptable. Intra-assay variation CV % was accepted below 10% and inter-assay variation was accepted below 15%.
CV %, coefficient of variance percentage; LLOQ, lower limit of quantification; LOB, limit of blank; ULOQ, upper limit of quantification; REC %, recovery percentage; MRD, minimum required dilution; h, hours; C., Celsius.

### Clinical Evaluation of FN-ANA and Comparison to FN-EDB:

After assay development and technical evaluation had been finalized, the clinical evaluation of FN-ANA was conducted in a cohort of 98 prevalent IPF patients and 133 healthy control subjects with demographics summarized in Table 3.

TABLE 3
Demographics table for healthy control and IPF subjects

| | | | Healthy |
|---|---|---|---|
| Parameters | IPF | | controls |
| n | 98 | | 133 |
| Age, median (IQR) | 73 (68-76.3) | yrs | 36 (29-49) yrs |
| Male sex, n (percentage) | 81 | (80.2%) | 66 (49.3%) |
| BMI, mean (SD) | 26.2 | (±3.9) | — |
| Smoking status, n | | | — |
| (percentage) | | | |
| Never | 29 | (29.6%) | |
| Former | 64 | (65.3%) | |
| Current | 5 | (5.1%) | |
| Time since diagnosis, mean | 21.1 (20.9) | months | — |
| (SD) | | | |
| FVC (% of predicted), | 83.5% | (±24.2%) | — |
| mean (SD) | | | |
| DLCO (% of predicted), | 46.4% | (±14.4%) | — |
| mean (SD) | | | |
| 6MWD, mean (SD) | 447.8 m | (±125.2 m) | — |
| Antifibrotic treatment, n | | | — |
| (percentage) | | | |
| Pirfenidone | 50 | (51.0%) | |
| Nintedanib | 31 | (31.6%) | |
| No treatment | 17 | (17.4%) | |

BMI, body mass index;
DLCO, diffusing capacity for carbon monoxide percent predicted,
FVC, forced vital capacity;
IQR, inter quartile range;
m, meters;
SD, standard deviation;
6MWD, six-minute walk distance

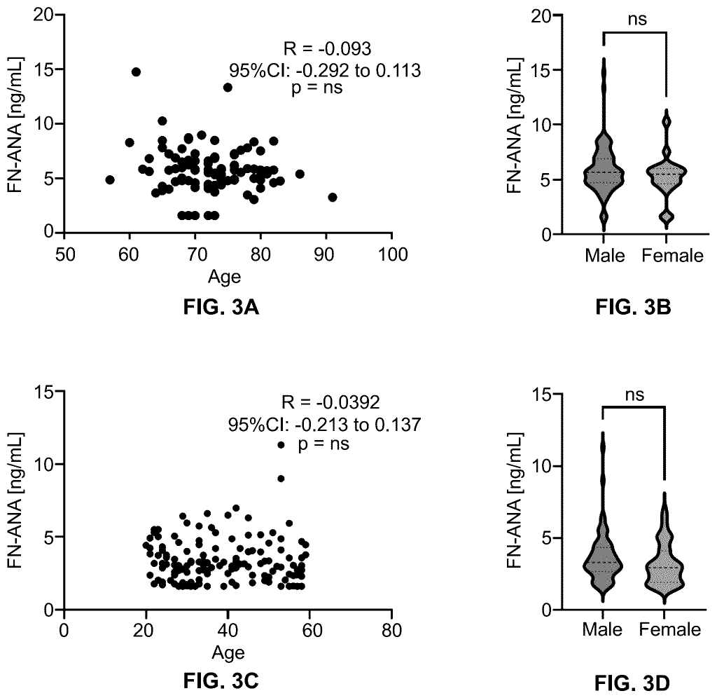

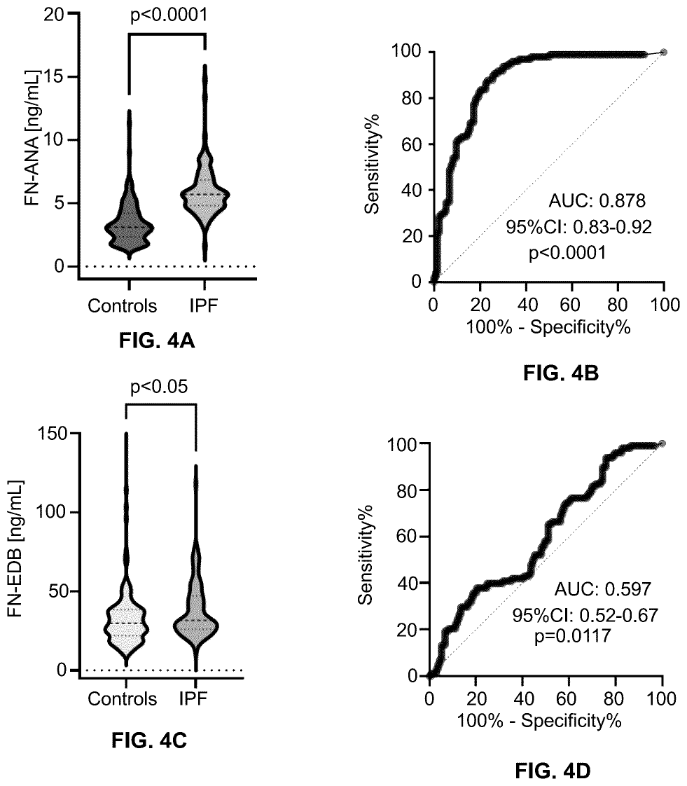

No association of FN-ANA was found for age or sex in healthy control or IPF subjects (FIGS. 3A-3D). Serum FN-ANA levels were significantly elevated in IPF patients (median 5.68 (IQR 4.80-6.83) ng/ml) when compared to healthy control subjects (median 3.09 (IQR 2.34-4.21) ng/ml; p<0.0001) (FIG. 4A). The separation between the groups was further investigated by ROC curve analysis that showed good separation of IPF and healthy controls with an AUC of 0.878 (95% CI 0.83-0.92, p<0.0001) (FIG. 4B). In comparison, FN-EDB serum levels were also significantly different when comparing healthy control (median 29.7 (IQR 21.72-38.24) ng/ml) to IPF (median 31.38 (IQR 25.79-46.84) ng/ml, p=0.0116) (FIGS. 4C-4D) but to a lesser extent than FN-ANA and with very similar median levels for the two groups. Accordingly, the ROC curve analysis achieved statistical significance when separating healthy controls from IPF subjects but with a lower AUC of 0.598 (95% CI 0.52-0.67, p=0.0117).

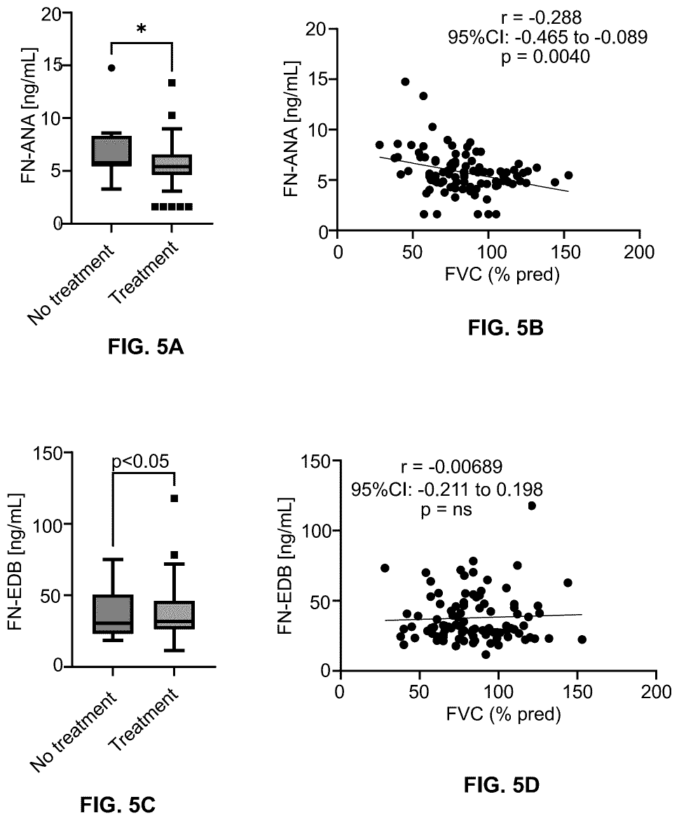

FN-ANA serum levels were significantly lower in IPF subjects who received antifibrotic treatment (mean 5.55 ng/ml) when compared to those who did not (mean 6.78 ng/ml, p=0.0471) (FIG. 5A). Additionally, FN-ANA serum levels were negatively correlated with the clinically relevant lung function measure FVC (FVC; r=−0.288; p=0.0040) (FIG. 5B). Contrary to this, FN-EDB serum levels were not significantly different between IPF subjects on (mean 38.24 ng/ml) or off (mean 37.69 ng/ml, p=ns) antifibrotic treatment, nor did levels correlate with FVC (FVC; r=−0.007, p=ns) (FIGS. 5C-5D).

### Discussion

A novel competitive chemiluminescent immunoassay, the FN-ANA assay, has been developed that quantifies the free N-terminal anastellin neoepitope in serum. The assay is specific to this neoepitope, accurate, precise, and stable. In addition, FN-ANA is a relevant biomarker for IPF.

Increased expression and deposition of different fibronectin domains has been associated with fibrosis previously (15-17). The anastellin domain has not previously been quantified in serum or proposed as a biomarker for IPF. The serological biomarker FN-ANA was able to separate IPF and healthy subjects with higher efficacy than another type III domain biomarker of FN; FN-EDB (AUC of 0.878 vs. 0.598). It has previously been speculated that anastellin could be relevant as a potential treatment in cancer due to its inhibition of tumor growth, angiogenesis, and metastasis preclinically (7, 8, 10). The antifibrotic effects of anastellin have not been considered or shown in any preclinical or clinical setting previously nor its potential association with clinical parameters relevant for fibrosis. This study has shown that levels of circulating anastellin, assessed by the FN-ANA biomarker, were significantly associated with FVC, a commonly used endpoint in clinical trials of IPF. Interestingly, FN-EDB was not correlated with FVC levels. Additionally, FN-ANA may be a potential pharmacodynamic biomarker as serum levels were lower in IPF subjects receiving antifibrotic therapy, indicating a potential effect of treatment on circulating anastellin levels. In comparison, the FN-EDB biomarker did not show an association with treatment status, thus highlighting an advantage of FN-ANA over other FN type III domain biomarkers.

In conclusion, the invention provides a novel method for quantifying circulating anastellin in serum by measuring a unique fragment of the free N-terminal of Anastellin. The FN-ANA assay is robust and provides a biomarker for IPF with diagnostic and pharmacodynamic capabilities as well as associations to disease severity determined by lung function measures, outperforming another FN biomarker.

### REFERENCES

1. Maher et al., Respir Res, 22(1):197, July 2021.
2. Martinez et al., Nat Rev Dis Primers, 3(1):17074, 2017.
3. C. J. Dalton and C. A. Lemmon, Cells, 10(9). 2021.
4. Baneyx et al. Proceedings of the National Academy of Sciences, 99(8):5139-5143, 2002.
5. Cho et al. Curr Top Pept Protein Res, 16:37, 2015.
6. Morla et al. J Cell Biol, 118(2):421, July 1992.
7. A. Ambesi and P. J. Mckeown-Longo, Molecular Cancer Research, 7(2):255-265, 2009.
8. M. Yi and E. Ruoslahti, Proceedings of the National Academy of Sciences, 98(2):620-624, 2001.
9. Pasqualini et al. Nat Med, 2(11):1197-1203, 1996.
10. Neskey et al. Journal of Experimental & Clinical Cancer Research, 27:1-8, 2008.
11. Combet et al. Trends Biochem Sci, 25(3):147-150, March 2000.
12. Bateman et al. Nucleic Acids Res, 45(D1):D158-D169, January 2017.
13. Gefter et al. Somatic Cell Genet, 3(2):231-236, March 1977.
14. Prior et al. Respir Med, Vol. 185, August 2021.
15. Muro et al. Am J Respir Crit Care Med, 177(6):638-645, 2008.
16. Novak et al. American Journal of Physiology-Lung Cellular and Molecular Physiology, 324(4):L507-L520, 2023.
17. Upagupta et al. European Respiratory Review, 27(143), 2018.

What is claimed is:

1. A monoclonal antibody that specifically recognises the N-terminus amino acid sequence PIQWNAPQPS (SEQ ID NO: 1).

2. The monoclonal antibody of claim 1, wherein the monoclonal antibody specifically binds to the N-terminus amino acid sequence PIQWNAPQPS (SEQ ID NO: 1) and does not specifically bind to a peptide having the N-terminus amino acid sequence HPIQWNAPQPS (SEQ ID NO: 2).

3. The monoclonal antibody of claim 1, wherein the monoclonal antibody specifically binds to the N-terminus amino acid sequence PIQWNAPQPS (SEQ ID NO: 1) and does not specifically bind to a peptide having the N-terminus amino acid sequence IQWNAPQPS (SEQ ID NO: 3).

4. The monoclonal antibody of claim 1, wherein the monoclonal antibody is raised against a synthetic peptide having the N-terminus amino acid sequence PIQWNAPQPS (SEQ ID NO: 1).

5. A method of immunoassay, said method comprising;
i) contacting a patient sample with a monoclonal antibody that specifically binds to the N-terminus amino acid sequence PIQWNAPQPS (SEQ ID NO: 1); and
ii) detecting and determining the amount of binding between said monoclonal antibody and peptides in the sample.

6. The method as claimed in claim 5, wherein the method is a method of immunoassay for detecting and/or monitoring a fibrotic disease or a particular level of severity thereof in a patient, the method further comprising;
iii) correlating said amount of binding with values associated with normal healthy subjects and/or values associated with known disease severity and/or values obtained from said patient at a previous time point and/or with a predetermined cut-off value.

7. The method as claimed in claim 6, wherein the fibrotic disease is idiopathic pulmonary fibrosis.

8. The method of claim 5, wherein the patient sample is selected from blood, plasma or serum.

9. The method of claim 5, wherein the monoclonal antibody specifically binds to the N-terminus amino acid sequence PIQWNAPQPS (SEQ ID NO: 1) and does not specifically bind to a peptide having the N-terminus amino acid sequence HPIQWNAPQPS (SEQ ID NO: 2).

10. The method of claim 5, wherein the monoclonal antibody specifically binds to the N-terminus amino acid sequence PIQWNAPQPS (SEQ ID NO: 1) and does not specifically bind to a peptide having the N-terminus amino acid sequence IQWNAPQPS (SEQ ID NO: 3).

11. The method of claim 5, wherein the monoclonal antibody is raised against a synthetic peptide having the N-terminus amino acid sequence PIQWNAPQPS (SEQ ID NO: 1).

12. The method of claim 5, wherein the immunoassay is a competition assay or a sandwich assay.

13. The method of claim 5, wherein the immunoassay is a radio-immunoassay or an enzyme-linked immunosorbent assay.

14. An immunoassay kit, comprising:
the monoclonal antibody of claim 1, and
at least one of:
a streptavidin coated well plate;
a biotinylated peptide PIQWNAPQPS-L-Biotin (SEQ ID NO: 4), wherein L is an optional linker;
a secondary antibody for use in a sandwich immunoassay;
a calibrator protein comprising the N-terminus amino acid sequence PIQWNAPQPS (SEQ ID NO: 1);
an antibody biotinylation kit;
an antibody HRP labelling kit; or
an antibody radiolabelling kit.
